(19) United States
(12) Patent Application Publication
(10) Pub. No.: US 2024/0252043 A1
(43) Pub. Date: Aug. 1, 2024

(54) BODY TEMPERATURE ESTIMATING DEVICE AND BODY TEMPERATURE ESTIMATING METHOD

(71) Applicant: NIPPON TELEGRAPH AND TELEPHONE CORPORATION, Tokyo (JP)

(72) Inventors: Shin TOYOTA, Musashino-shi (JP); Kazuyoshi ONO, Musashino-shi (JP)

(73) Assignee: NIPPON TELEGRAPH AND TELEPHONE CORPORATION, Tokyo (JP)

(21) Appl. No.: 18/565,833

(22) PCT Filed: Jun. 8, 2021
(86) PCT No.: PCT/JP2021/021683

§ 371 Date: Nov. 30, 2023

(51) Int. Cl. A61B 5/01; A61B 5/00

(52) U.S. Cl. CPC A61B 5/01; A61B 5/0059; A61B 5/6803

(57) ABSTRACT

A body temperature estimating device includes a visible light transmissive member, a wearing member, a sensor, and a processing unit. The visible light transmissive member transmits visible light and reflects infrared light. The wearing member is worn by a wearer. The wearing member holds the visible light transmissive member so as to be disposed in front of eyes of the wearer. The sensor detects infrared light emitted from the wearer of the wearing member and reflected by the visible light transmissive member. The processing unit estimates a body temperature of the wearer on the basis of the infrared light detected by the sensor.

### TECHNICAL FIELD

The present invention relates to a body temperature estimating device and a body temperature estimating method.

### BACKGROUND ART

There is a body temperature estimating device that captures a thermal image by using infrared thermography and estimates a body temperature from the obtained thermal image. The body temperature estimating device using the thermal image can measure the body temperature in a non-contact manner and instantly. Such a body temperature estimating device is installed at an entrance of a commercial facility or the like, and is utilized for countermeasures against a pandemic (see, for example, Non Patent Literature 1).

In a case where body temperature measurement is performed by utilizing infrared thermography under a situation where a line of flow of people is not fixed, such as when the body temperature estimating device is introduced at an entrance of a commercial facility or the like, there is a possibility that there is a people who is not a detection target of body temperature rise because a measurement range is limited. On the other hand, there is an eyeglass-type wearable device (see, for example, Non Patent Literature 2).

### CITATION LIST

### Non Patent Literature

Non Patent Literature 1: Jiro Ota, Eri Hamada, “Introduction of example of body surface temperature measurement by infrared thermography from viewpoint of preventing influenza from spreading”, NEC Technical Journal, Vol. 62 No. 3, 2009, pp. 87-91
Non Patent Literature 2: “Utilizing eyeglass-type wearable terminal “b.g.” for remote training”, [online], Mar. 26, 2020, VISIONARY HOLDINGS CO., LTD., Internet <URL: https://www.visionaryholdings.co.jp/wp/wp-content/uploads/2020/03/wearable_inflection20200326_2.pdf>

### SUMMARY OF INVENTION

### Technical Problem

A body temperature of a wearer can be measured by attaching a sensor for body temperature measurement to an eyeglass-type wearable device. However, when the sensor is disposed in front of the eyeglass-type wearable device, there is a problem that a part of a field of view of the wearer is obstructed.

In view of the above circumstances, an object of the present invention is to provide a wearable body temperature estimating device and a body temperature estimating method for estimating a body temperature not to obstruct a field of view as much as possible.

### Solution to Problem

A body temperature estimating device of an aspect of the present invention includes: a visible light transmissive member that transmits visible light and reflects infrared light; a wearing member that is worn by a wearer and holds the visible light transmissive member so as to be disposed in front of eyes of the wearer; a sensor that detects infrared light emitted from the wearer and reflected by the visible light transmissive member; and a processing unit that estimates a body temperature of the wearer on the basis of the infrared light detected by the sensor.

A body temperature estimating method of an aspect of the present invention includes an estimation step of detecting infrared light and estimating a body temperature of a wearer wearing a wearing member on a basis of the infrared light detected, in which the wearing member holds a visible light transmissive member, that transmits visible light, so as to be disposed in front of eyes of the wearer, and the infrared light is emitted from the wearer and reflected by the visible light transmissive member.

### Advantageous Effects of Invention

According to the present invention, it is possible to estimate the body temperature by the wearable body temperature estimating device not to obstruct the field of view as much as possible.

### BRIEF DESCRIPTION OF DRAWINGS

FIG. 1 is a diagram illustrating a configuration example of an eyeglass-type wearable device according to a first embodiment of the present invention.

FIG. 2 is a diagram illustrating the vicinity of an eye of a wearer of the eyeglass-type wearable device according to the embodiment.

FIG. 3 is a diagram illustrating a configuration example of a frame unit according to the embodiment.

FIG. 4 is a diagram illustrating a configuration example of a processing unit according to the embodiment.

FIG. 5 is a flow diagram illustrating processing performed by the processing unit according to the embodiment.

FIG. 6 is a diagram illustrating a hardware configuration of the frame unit according to the embodiment.

FIG. 7 is a diagram illustrating a configuration example of a body temperature estimating device according to a second embodiment.

FIG. 8 is a diagram illustrating an example in which the body temperature estimating device according to the embodiment is worn.

### DESCRIPTION OF EMBODIMENTS

Hereinafter, embodiments of the present invention will be described in detail with reference to the drawings.

### First Embodiment

A body temperature estimating device according to a first embodiment is an eyeglass-type body temperature estimating device. The eyeglass-type body temperature estimating device reflects an optical path of infrared light emitted from a face surface of a wearer of the device by an eyeglass lens. In the eyeglass-type body temperature estimating device, an infrared sensor is disposed not to obstruct a field of view of the wearer as much as possible. The infrared light reflected by the eyeglass lens is incident on the infrared sensor. The eyeglass-type body temperature estimating device estimates a body temperature of the wearer on the basis of a detection value by the infrared sensor.

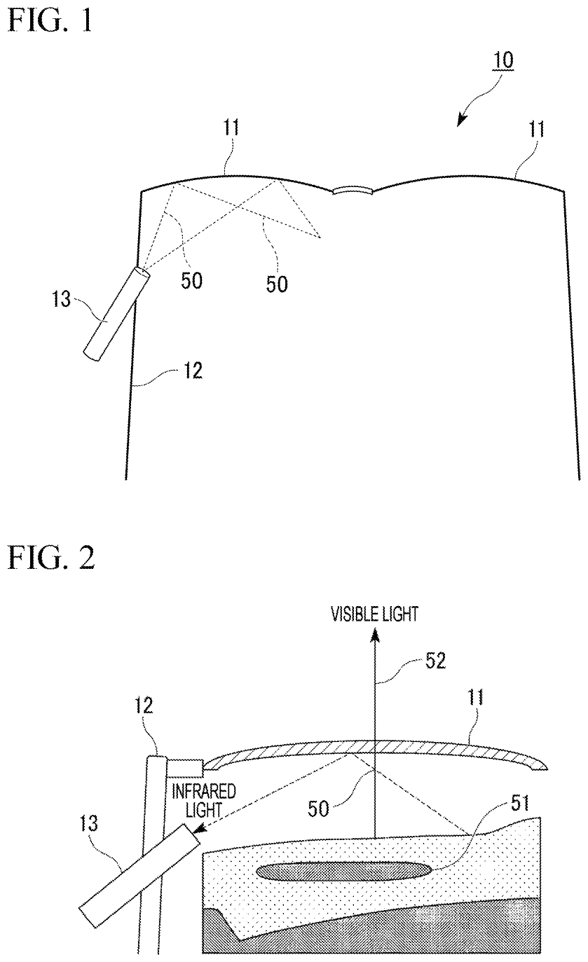

FIG. 1 is a diagram illustrating a configuration example of an eyeglass-type wearable device 10 of the present embodiment. The eyeglass-type wearable device 10 is an example of the body temperature estimating device. The eyeglass-type wearable device 10 includes an eyeglass lens 11 and an eyeglass frame 12.

The eyeglass lens 11 is a lens that reflects infrared light. For example, the eyeglass lens 11 is obtained by applying dielectric multilayer film coating to a generally used lens for eyeglasses. As an example of the dielectric multilayer film coating, there is a method of using a dichroic filter. The eyeglass lens 11 may be obtained by applying or sticking a transparent material that reflects infrared light to a general lens for eyeglasses. Examples of such a material include paints, films, ultrathin metal films, and the like. Here, the eyeglass lens 11 is a lens for eyeglasses on which an infrared reflecting mirror is deposited. The infrared reflecting mirror reflects infrared light and transmits visible light.

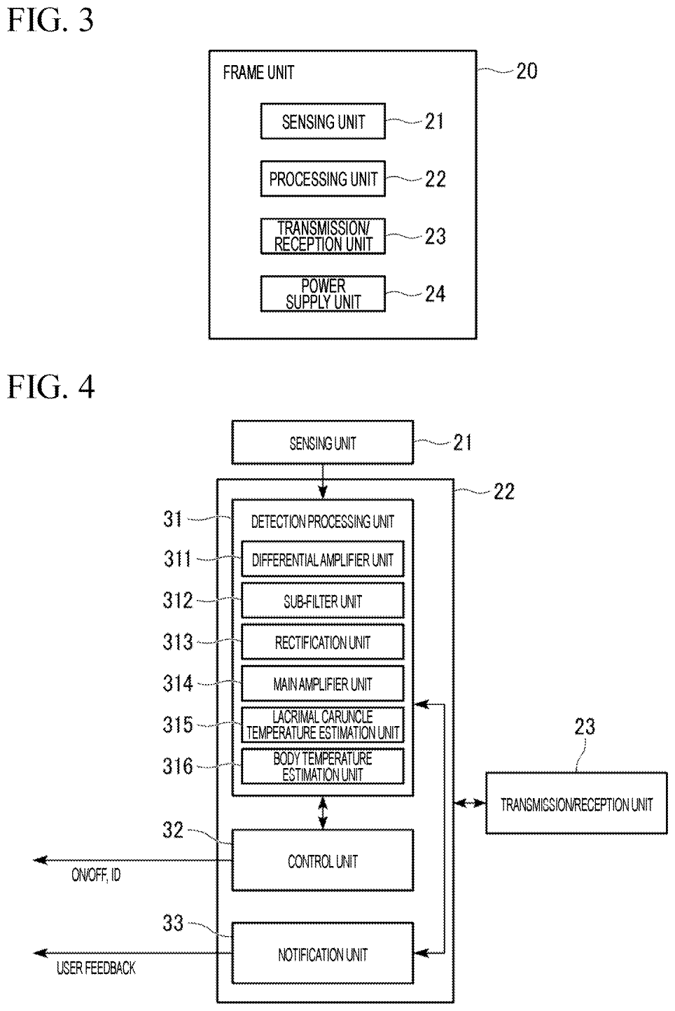

The eyeglass frame 12 is obtained by including a frame unit 20 illustrated in FIG. 3 described later in a general eyeglass frame. The eyeglass frame 12 holds the eyeglass lens 11. An infrared sensor 13 is disposed on the eyeglass frame 12. The infrared sensor 13 is disposed on a Tsuru (in Japanese) of the eyeglass frame 12, for example. The Tsuru is also called a temple, and is a portion for putting eyeglasses on the ear of the wearer. The infrared sensor 13 is a sensor that detects infrared light. Infrared light 50 reflected by the eyeglass lens 11 is incident on the infrared sensor 13.

FIG. 2 is a diagram illustrating the vicinity of a left eye 51 of the wearer of the eyeglass-type wearable device 10. The eyeglass lens 11 transmits visible light 52 and reflects the infrared light 50. The infrared light 50 reflected by the eyeglass lens 11 is incident on the infrared sensor 13 disposed on the temple of the eyeglass frame 12. By disposing the infrared sensor 13 on the temple, it is possible to sense the infrared light 50 emitted from the face surface of the wearer while preventing the field of view of the wearer from being obstructed. In addition, by disposing the infrared sensor 13 on the temple of the eyeglass frame 12, it is possible for the infrared sensor 13 to acquire the infrared light 50 at a lacrimal caruncle portion of the wearer from reflection by the eyeglass lens 11. Since a temperature of the lacrimal caruncle portion is a high value among face surface temperatures, the body temperature can be estimated by obtaining a lacrimal caruncle temperature. As a result, it is possible to estimate the body temperature of the wearer without blocking the field of view of the wearer as much as possible while the eyeglass-type wearable device 10 is worn.

FIG. 3 is a diagram illustrating a configuration of the frame unit 20. The frame unit 20 is included in the eyeglass frame 12 illustrated in FIG. 1. The frame unit 20 includes a sensing unit 21, a processing unit 22, a transmission/reception unit 23, and a power supply unit 24.

The sensing unit 21 is, for example, the infrared sensor 13 illustrated in FIGS. 1 and 2. The sensing unit 21 is a sensor that measures infrared light from an inner corner of an eye of the wearer reflected by the infrared reflecting mirror of the eyeglass lens 11. The sensing unit 21 adds a sensor ID to data obtained by measurement and outputs the data to the processing unit 22. The sensor ID is information for identifying a sensor used as the sensing unit 21.

The processing unit 22 performs signal processing on the data received from the sensing unit 21. The transmission/reception unit 23 wirelessly transmits and receives data to and from an external device. Note that the transmission/reception unit 23 may transmit and receive the data by wire. The transmission/reception unit 23 transmits the data processed by the processing unit 22 to an external device. The power supply unit 24 controls on/off of each unit of the frame unit 20.

FIG. 4 is a diagram illustrating a configuration example of the processing unit 22 illustrated in FIG. 3. The processing unit 22 includes a detection processing unit 31, a control unit 32, and a notification unit 33. The detection processing unit 31 performs signal processing on the data acquired by the sensing unit 21, and detects a temperature, a time, a sensor ID, and the like. The detection processing unit 31 includes a differential amplifier unit 311, a sub-filter unit 312, a rectification unit 313, a main amplifier unit 314, a lacrimal caruncle temperature estimation unit 315, and a body temperature estimation unit 316.

The differential amplifier unit 311 amplifies a signal. The sub-filter unit 312 removes noise from the signal amplified by the differential amplifier unit 311. The rectification unit 313 shapes a waveform of the signal from which the noise has been removed by the sub-filter unit 312. The main amplifier unit 314 amplifies the signal whose waveform has been shaped by the rectification unit 313. The lacrimal caruncle temperature estimation unit 315 acquires data of the lacrimal caruncle temperature from the signal. The body temperature estimation unit 316 estimates the body temperature from the lachrymal temperature acquired by the lacrimal caruncle temperature estimation unit 315.

The control unit 32 refers to an on/off state of the processing unit 22 by the power supply unit 24 and the sensor ID detected by the detection processing unit 31, and controls the detection processing unit 31 on the basis of a result of referring. The notification unit 33 transmits data detected by the detection processing unit 31 and data indicating success or failure of detection by the detection processing unit 31 from the transmission/reception unit 23. In addition, the notification unit 33 feeds back information indicating success or failure of detection by the detection processing unit 31 to a user. For example, sound output, lighting of a lamp, display on a display, or the like can be used as feedback, but the feedback is not limited thereto.

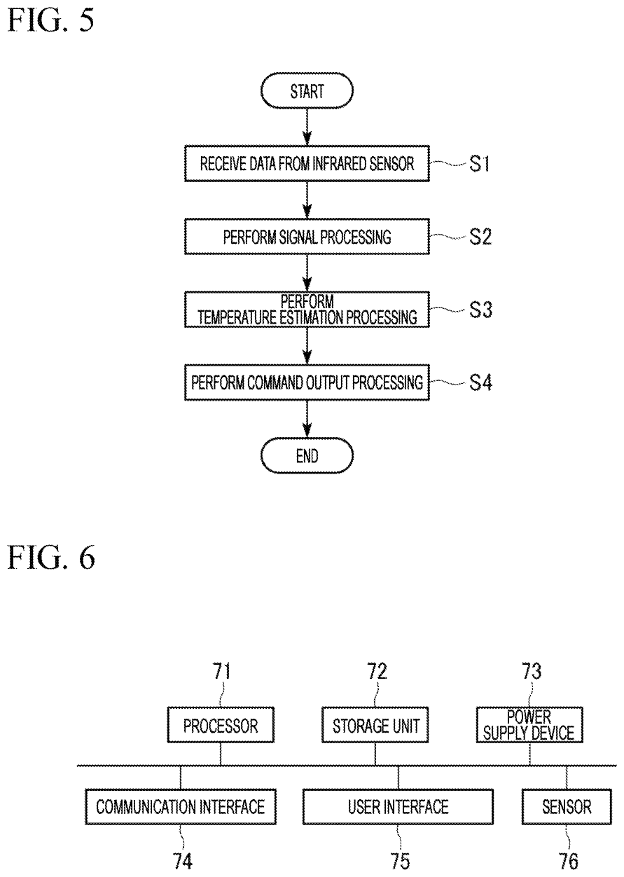

FIG. 5 is a flow diagram illustrating an example of body temperature detection processing performed by the processing unit 22. The infrared sensor 13 outputs data in which a sensor value, a sensor ID, a detection time of the sensor value, and the like are set. The detection processing unit 31 receives the data output from the infrared sensor 13 (step S1), and performs signal processing on the received data (step S2). Specifically, the differential amplifier unit 311 amplifies the signal, and the sub-filter unit 312 removes noise from the amplified signal. The rectification unit 313 shapes the waveform of the signal from which the noise has been removed, and the main amplifier unit 314 amplifies the waveform-shaped signal.

The detection processing unit 31 estimates the body temperature by using information obtained from the data subjected to the signal processing (step S3). Specifically, the lacrimal caruncle temperature estimation unit 315 acquires a sensor value from the data subjected to the signal processing in step S2, and acquires information on the lacrimal caruncle temperature on the basis of the acquired sensor value. The body temperature estimation unit 316 estimates the body temperature on the basis of the lacrimal caruncle temperature acquired by the lacrimal caruncle temperature estimation unit 315.

The control unit 32 performs command output processing for outputting information on the body temperature estimated by the detection processing unit 31 in step S3 to the outside (step S4). As a result, the notification unit 33 transmits the information on the body temperature estimated to an external device in accordance with an instruction from the control unit 32. The notification unit 33 may transmit the information on the body temperature by adding information on the time and the sensor ID acquired by the detection processing unit 31. The external device outputs the received information on the body temperature, the time, and the sensor ID by displaying the information on a display or the like. In addition, the detection processing unit 31 feeds back success of estimation of the body temperature to the user.

FIG. 6 is a diagram illustrating a hardware configuration example of the frame unit 20. The frame unit 20 includes a processor 71, a storage unit 72, a power supply device 73, a communication interface 74, a user interface 75, and a sensor 76. The processor 71 is a central processing device that performs calculation and control. The processor 71 is a central processing unit (CPU), for example. The processor 71 reads a program from the storage unit 72, and executes the program. The storage unit 72 further includes a work area and the like to be used when the processor 71 executes various programs. The power supply device 73 supplies power to each unit. The communication interface 74 is for communicable connection to another device. The user interface 75 is an input device such as a button or a display device such as a lamp or a display. The sensor 76 is a sensor that detects infrared light.

All or some of functions of the processing unit 22 are implemented by the processor 71 reading a program from the storage unit 72 and executing the program. Note that all or some of these functions may be implemented by using hardware such as an application specific integrated circuit (ASIC), a programmable logic device (PLD), or a field programmable gate array (FPGA).

When eyeglasses are worn, infrared light on the face surface of the wearer cannot be sensed by a thermography camera or the like due to eyeglass lenses. Thus, it is conceivable to perform infrared sensing by attaching an infrared sensor to an eyeglass device. However, in the eyeglass device, it is important to secure a field of view of the wearer. If the infrared sensor is attached to the front of the eyeglass device, a line of sight of the wearer is obstructed. Thus, the eyeglass-type wearable device 10 of the present embodiment reflects infrared light by the eyeglass lens 11 subjected to dielectric multilayer film coating. Furthermore, the infrared sensor 13 is disposed such that an optical path of infrared light is reflected by the eyeglass lens 11 and is incident on the infrared sensor 13. Since humans cannot feel infrared light, infrared light can be sensed without obstructing visible light, and the body temperature can be estimated on the basis of the sensed infrared light.

### Second Embodiment

A body temperature estimating device of a second embodiment is a face shield type body temperature estimating device. The present embodiment will be described focusing on a difference from the first embodiment.

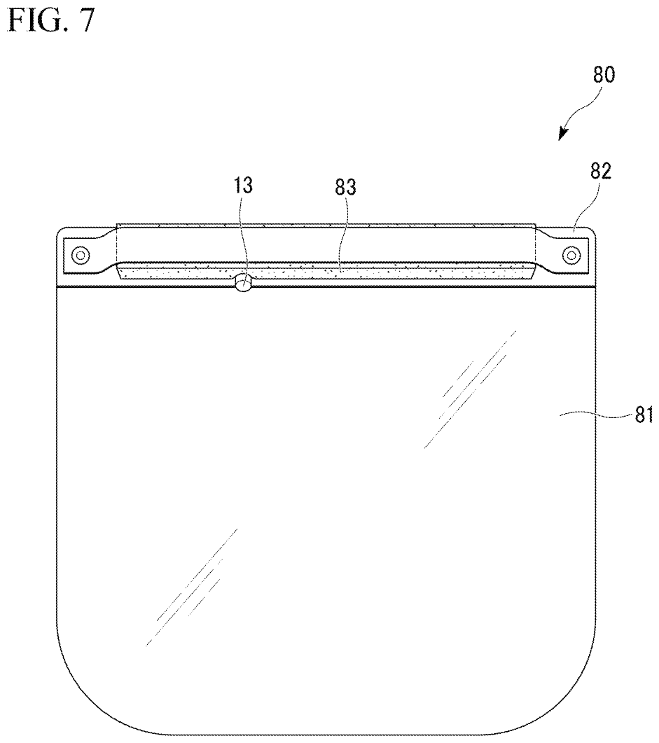

FIG. 7 is a diagram illustrating a configuration example of a body temperature estimating device 80. FIG. 7 illustrates a configuration when viewed from a surface to be worn on the head. The body temperature estimating device 80 includes a shield 81 and a frame 82. The shield 81 covers a part or whole of the face of the wearer. The shield 81 transmits visible light and reflects infrared light. The shield 81 is obtained by applying dielectric multilayer film coating to a shield of a generally used face shield similarly to the eyeglass lens 11 of the first embodiment. In addition, the shield 81 may be formed by applying or sticking a transparent material that reflects infrared light to a shield of a general face shield similarly to the eyeglass lens 11 of the first embodiment.

The frame 82 is a member for causing the body temperature estimating device 80 to be worn on the head of the wearer. The frame 82 is obtained by including the frame unit 20 of the first embodiment illustrated in FIG. 3 in a frame of a general face shield. The frame 82 holds the shield 81 such that the shield 81 is disposed in front of the eyes or face of the wearer. The infrared sensor 13 of the frame unit 20 is disposed on the frame 82. The infrared sensor 13 is disposed, for example, on a forehead pad 83 of the frame 82. The forehead pad 83 is a member serving as a cushion for bringing the frame 82 into close contact with the forehead of the wearer so that the frame 82 does not shift when the wearer wears the frame. The infrared light 50 reflected by the shield 81 is incident on the infrared sensor 13.

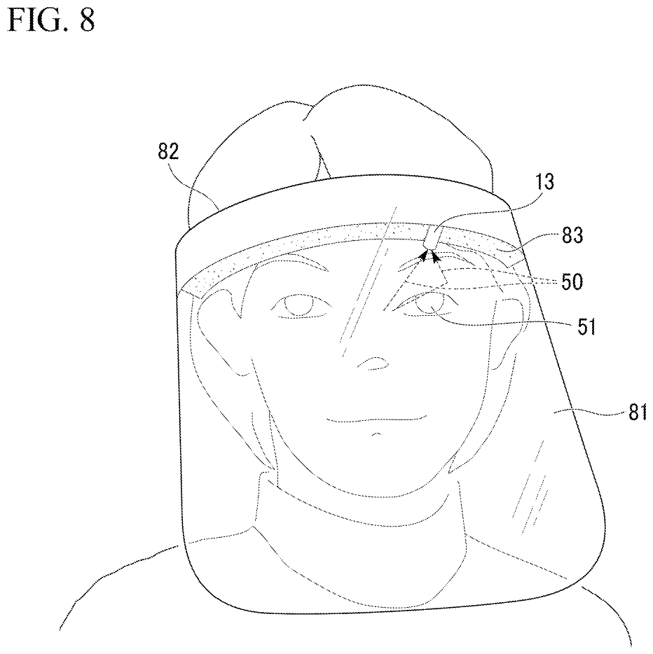

FIG. 8 is a diagram illustrating the wearer wearing the body temperature estimating device 80. The shield 81 reflects the infrared light 50 emitted from the face surface of the wearer. The infrared light 50 reflected by the shield 81 is incident on the infrared sensor 13 disposed on the forehead pad 83 of the frame 82. As a result, for the infrared light 50 from the left eye 51, there are a case where the infrared light is incident on the infrared sensor 13 in a straight line and a case where the infrared light is reflected by the shield 81 and then incident on the infrared sensor 13. The frame unit 20 operates similarly to the first embodiment.

By disposing the infrared sensor 13 on the frame 82 worn on the head above the eyes, it is possible to acquire the infrared light 50 emitted from the face of the wearer from reflection by the shield 81 while preventing the field of view of the wearer from being obstructed. In particular, by disposing the infrared sensor 13 in the forehead pad 83 of the frame 82, it is possible to acquire the infrared light 50 emitted from the lacrimal caruncle portion of the wearer directly or from reflection by the shield 81. Thus, it is possible to estimate the body temperature of the wearer without blocking the field of view of the wearer as much as possible while the body temperature estimating device 80 is worn.

According to the embodiments described above, the body temperature estimating device includes a visible light transmissive member, a wearing member, a sensor, and a processing unit. The visible light transmissive member transmits visible light and reflects infrared light. For example, the visible light transmissive member is a transparent member. Here, the term “transparent” means that at least a part of visible light is transmitted. For example, the visible light transmissive member is subjected to dielectric multilayer film coating. The wearing member is worn by a wearer. For example, the wearing member is worn on the head of the wearer. The wearing member holds the visible light transmissive member so as to be disposed in front of eyes of the wearer. The sensor detects infrared light emitted from the wearer and reflected by the visible light transmissive member. The processing unit estimates a body temperature of the wearer on the basis of the infrared light detected by the sensor.

The body temperature estimating device is, for example, an eyeglass-type body temperature estimating device. The eyeglass-type body temperature estimating device includes an eyeglass lens, an eyeglass frame, a sensor, and a processing unit. The eyeglass frame holds the eyeglass lens. The sensor detects infrared light emitted from a wearer of the eyeglass frame and reflected by the eyeglass lens. The processing unit estimates a body temperature of the wearer on the basis of the infrared light detected by the sensor. For example, the sensor is disposed on a temple of the eyeglass frame.

In addition, the body temperature estimating device is, for example, a face shield type body temperature estimating device. In this case, the visible light transmissive member is a shield that covers at least a part of the face of the wearer, and the wearing member is a frame worn on the head above the eyes of the wearer. The sensor is disposed, for example, at a position near the forehead of the frame.

Although the embodiments of the present invention have been described in detail with reference to the drawings so far, specific configurations are not limited to these embodiments, and include designs and the like without departing from the gist of the invention.

### REFERENCE SIGNS LIST

10 eyeglass-type wearable device
11 eyeglass lens
12 eyeglass frame
13 infrared sensor
20 frame unit
21 sensing unit
22 processing unit
23 transmission/reception unit
24 power supply unit
31 detection processing unit
32 control unit
33 notification unit
71 processor
72 storage unit
73 power supply device
74 communication interface
75 user interface
76 sensor
80 body temperature estimating device
81 shield
82 frame
83 forehead pad
311 differential amplifier unit
312 sub-filter unit
313 rectification unit
314 main amplifier unit
315 lacrimal caruncle temperature estimation unit
316 body temperature estimation unit

1. A body temperature estimating device comprising:
a visible light transmissive member that transmits visible light and reflects infrared light;
a wearing member that is worn by a wearer and holds the visible light transmissive member so as to be disposed in front of eyes of the wearer;
a sensor that detects infrared light emitted from the wearer and reflected by the visible light transmissive member; and
a processor that estimates a body temperature of the wearer on a basis of the infrared light detected by the sensor.

2. The body temperature estimating device according to claim 1, wherein
the visible light transmissive member is an eyeglass lens, and
the wearing member is an eyeglass frame.

3. The body temperature estimating device according to claim 2, wherein
the sensor is disposed on a temple of the eyeglass frame.

4. The body temperature estimating device according to claim 1, wherein
the visible light transmissive member is a shield that covers at least a part of a face of the wearer, and
the wearing member is a frame worn on a head of the wearer.

5. The body temperature estimating device according to claim 4, wherein
the sensor is disposed on the frame.

6. The body temperature estimating device according to claim 1, wherein
the visible light transmissive member is subjected to dielectric multilayer film coating.

7. A body temperature estimating method comprising:
detecting infrared light emitted from a wearer wearing a wearing member, the wearing member holding a visible light transmissive member, that transmits visible light, so as to be disposed in front of eyes of the wearer, and reflected by the visible light transmissive member; and
estimating a body temperature of the wearer.
